(19) United States
(12) Patent Application Publication
(10) Pub. No.: US 2023/0218681 A1
(43) Pub. Date: Jul. 13, 2023

(54) BREVUNDIMONAS SP FOR USE IN DISEASE PREVENTION AND TREATMENT

(71) Applicant: Zivo Bioscience, Inc., Bloomfield Hills, MI (US)

(72) Inventors: Amy E. Steffek, Bloomfield Hills, MI (US); William P. Pfund, Bloomfield Hills, MI (US); Andrew A. Dahl, Bloomfield Hills, MI (US)

(73) Assignee: Zivo Bioscience, Inc., Bloomfield Hills, MI (US)

(21) Appl. No.: 18/077,132

(22) Filed: Dec. 7, 2022

Related U.S. Application Data
(60) Provisional application No. 63/286,599, filed on Dec. 7, 2021.
(60) Provisional application No. 63/355,799, filed on Jun. 27, 2022.

(51) Int. Cl. A61K 35/74; A61P 33/02; A61K 31/7028; A23K 10/16; A23K 50/75; A23K 20/163

(52) U.S. Cl. CPC A61K 35/74; A61P 33/02; A61K 31/7028; A23K 10/16; A23K 50/75; A23K 20/163

(57) ABSTRACT

An improved agent and treatment method for a broad variety of diseases in both animals and humans is disclosed. The agent is an inventive composition comprising a bacterial-based culture. Particularly, the inventive composition disclosed herein is an active molecule produced by a Gram-negative bacterial strain that is a member of the genus Brevundimonas. Consumption of the inventive composition by way of liquid or dry feed produces a broad range of health benefits and has proven effective in the prevention and treatment of disease. The bacterium of the disclosed composition selectively modulates Toll-like receptors (TLRs) for the prevention and treatment of disease such as coccidiosis. Specific actions include but are not limited to improvement of gut health, acceleration or enhancement of immune response using a natural immune modulator, and the promotion of animal growth.

### CROSS REFERENCE TO RELATED APPLICATIONS

This application claims priority to U.S. Provisional Patent Application Ser. No. 63/286,599 filed Dec. 7, 2021, and to U.S. Provisional Patent Application Ser. No. 63/355,799 filed Jun. 27, 2022, which are incorporated herein by reference in their entireties.

### TECHNICAL FIELD

The present invention relates to the use of a bacteria-based compound for the prevention and treatment of disease via oral intake by way of feed or drinking water or both. More particularly, the present invention relates to a compound and the use of a compound such as that derived from an active compound of Gram-negative bacterial strains that are members of the genus Brevundimonas. The aforementioned compound selectively modulates Toll-like receptors (TLRs) for the prevention and treatment of disease such as coccidiosis, necrotic enteritis, and other pathogenic infections via a direct effect on innate and adaptive immune pathways. Specific benefits of the compound and its use include but are not limited to improvements to gut health, acceleration or enhancement of the immune response, immune modulation, and the promotion of animal growth.

### BACKGROUND OF THE INVENTION

The overall health of the digestive tract of all animals, and specifically in poultry, is of critical concern in many industries. Poor digestive health results in lowered production rates and increased morality in production animals. Positive gut health is directly related to the overall performance, health and welfare of animals used in the food production industry. Gut health is itself highly complex, depending as it does on a variety of factors, including nutrition and immunology.

Negative gut health generally interferes with the normal conversion of animal feed intake into body weight gains. Gut morphology may be compromised by several factors, including disease and infection. Particularly susceptible to disease are the intestinal villi, surface area-expanding projections from the epithelial lining of the gut mucosa. Damage to gut morphology due to disease and infection compromises the ability of the intestine to absorb nutrients. In such a circumstance, feed utilization becomes less efficient, growth performance is jeopardized, and the risk of disease is increased.

The primary causative agents for gut disease include parasites and bacteria. Both types of agents take different courses in damaging gut health but each result in devastation to the animal as well as significant commercial loss.

The most common parasitic disease in poultry is coccidiosis. Caused by a protozoal parasite belonging to the genus Eimeria, coccidiosis may infect several locations in the intestine. Once introduced into the gut, the parasite quickly reproduces and, following a typical period of less than a week, causes damage to the intestinal mucosa and consequential loss of absorptive surface area. In poultry, a variety of coccidiosis-causing parasite species are known to exist, with each colonizing specific gut areas. The diseased birds suffer from an inability to absorb nutrients, in part, because of the morphologic change in the intestinal mucosa brought on by coccidiosis.

The most common bacterial disease in poultry is necrotic enteritis. Damage caused to the gut by coccidiosis may be a predisposing factor to the rapid onset of bacterial infection given that the intestinal mucosa is already compromised, leaving the animal susceptible to bacterial infection. It is thus very common for coccidiosis and necrotic enteritis to occur at the same time in a flock. An outbreak of necrotic enteritis in a flock may initially be overlooked as being little more than diarrhea and wet litter. Over time, typically by Day 35 of life, the flock shows signs of compromised nutrient absorption in response to failing gut health.

The bacterium responsible for necrotic enteritis, Clostridium perfringens, is commonly found in animal litter, feces, feed, soil, and dust. It may also be found in the gut of healthy poultry in low levels. Two types of Clostridium perfringens, types A and C, are ordinarily the causes of necrotic enteritis in poultry. Toxins generated by these bacterium types are known to damage the small intestine and create liver lesions in poultry.

It is thus desirable to develop an agent and method of treatment for coccidiosis in animals, particularly in poultry, thereby strengthening gut health. Not only will improved gut health of the animal reduce or eliminate the incidence of coccidiosis, but, as a consequence, the incidence of bacterial infection and resulting diseases to an already weakened gut, such as necrotic enteritis, will also be reduced.

### SUMMARY OF THE INVENTIONS

The disclosed inventive composition provides an improved agent and treatment method for a broad variety of diseases primarily in animals but may also find beneficial use in humans. The agent is an inventive composition comprising a bacterial-based material.

Particularly, the inventive composition disclosed herein is comprised of an active molecule of Gram-negative bacterial strains that are members of the genus Brevundimonas. Brevundimonas species are a non-fermenting, aerobic bacilli, which are commonly found in the environment but are not commonly isolated from clinical samples. The genus Brevundimonas is a member of the Caulobacterales order and the Caulobacteraceae family.

According to one exemplary embodiment, a composition is derived from the culture or co-culture of specific bacteria of ATCC Item No. SD-8636 received by the ATCC on or about Aug. 25, 2022.

In various embodiments, a method of preventing and treating disease in animals comprises administering to the animal a therapeutically effective amount of biomass from the culture or co-culture of specific bacteria of ATCC Item No. SD-8636 received by the ATCC on or about Aug. 25, 2022.

In various embodiments, a method of preventing or treating disease in animals comprises administering to the animal an effective amount of a composition derived from culturing microorganisms of ATCC Item No. SD-8636 received by the ATCC on or about Aug. 25, 2022. In various embodiments, the animal may be any one of cattle (bovines), pigs (porcines), sheep (ovines), goats (caprines), rabbits (leporines) or domesticated avians (such as, but not limited to, poultry).

The taxonomy of Brevundimonas is relatively complex and an understanding of the genus is ongoing as additional species are still being discovered. It is the very complexity of the genus which sometimes challenges identification: “For the 29 taxa identified among Brevundimonas genomes in GenBank in this study, it is possible that some are actually one of the twelve known Brevundimonas species but with no genome sequences of type strains being available. However, most of the 29 taxa (at least 17 taxa) appear to actually represent novel Brevundimonas species, which are unnamed as they have not been characterized by phenotype methods and therefore warrant further studies.” (https://journals.asm.org/doi/10.1128/Spectrum.00111-21)

Today thirty-four species of Brevundimonas are known, although additional species are continuously being added. Particular non-limiting examples of microbes classified in the genus Brevundimonas include: Brevundimonas alba, Brevundimonas aurantiaca, Brevundimonas bacteroides, Brevundimonas basaltis, Brevundimonas bullata, Brevundimonas diminuta, Brevundimonas intermedia, Brevundimonas kwangchunensis, Brevundimonas lenta, Brevundimonas mediterranea, Brevundimonas nasdae, Brevundimonas olei, Brevundimonas subvibrioides, Brevundimonas terrae, Brevundimonas vancanneytii, Brevundimonas variabilis, Brevundimonas vesicularis, and Brevundimonas viscosa.

While a number of species may be effectively incorporated into the present inventive composition, the preferred bacteria incorporated into the present inventive composition include Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis.

Brevundimonas vesicularis is one of two commonly-known human opportunistic pathogens of the Brevundimonas genus, the other being Brevundimonas diminuta. B. vesicularis can pose a challenge in the hospital setting as it survives in a nutrient-limited, damp environment.

Brevundimonas nasdae grows well under aerobic conditions at 30° on BHI agar plates. The bacterium is believed to be non-pathogenic in humans and has short, rod-shaped cells and produces circular and smooth colonies. B. nasdae utilizes acetate, pyruvate, methyl pyruvate, succinate and amino acids, including 1-alanine, 1-aspartic acid, glutamate and 1-proline, but not arabinose, mannose, fructose or lactose. B. nasdae produces acid from glucose, galactose, maltose and sucrose but is not able to hydrolyze Tween 80 or glycerol. (https://citeseerx.ist.psu.edu/viewdoc/download?doi=10.1.1.852.6666&rep=rep1&type=pdf)

Brevundimonas intermedia is commonly found in freshwater lakes. It is known to have a beneficial effect on plants as studies have shown that B. intermedia promoted growth in wheat plants. (https://pdfs.sem anticscholar.org/167a/00bfb08fb0bd559b5890fe7ad2b203e03bbf.pdf)

Brevundimonas aurantiaca is a bacterium that can produce catalase and oxidase enzymes when optimally grown at 45° C. B. aurantiaca has demonstrated an extreme arsenic tolerance having been isolated from an arsenic-rich hot spring and biologically absorbed arsenic inside the cell. A recent case study reported infective endocarditis (IE) caused by Brevundimonas aurantiaca infection in a human.

Brevundimonas mediterranea is a rod-shaped, non-spore-forming marine bacterium which has been isolated from salt water in the Mediterranean Sea. Since its first identification it has been isolated from 6582-m hadal sediment of the Pacific Ocean.

Brevundimonas albigilva is an aerobic, Gram-negative, short rod-shaped motile Proteobacterium which forms circular colonies. It was isolated from forest soil. It is classified as belonging to the genus Brevundimonas because of its phenotypic, genotypic, chemotaxonomic, and phylogenetic distinctiveness.

Brevundimonas huaxiensis is a Gram-negative, aerobic motile, non-spore forming, rod-shaped bacterium with an ability to assimilate sodium acetate. It does not utilize glucose, trypsin, or a β-glucosidase, thereby differentiating it phenotypically from other Brevundimonas species. Colonies are characterized as being circular, smooth, convex and orange. The strain was recovered from a hospital in West China.

Consumption of the inventive composition by way of liquid or dry feed produces a broad range of health benefits and has proven effective in the prevention and treatment of disease. Several actions have been realized which are set forth hereinafter.

I. Agents and Method for Improving Gut Health

The inventive composition disclosed herein has a positive impact on the gut health of boilers from the earliest stages of life and onward. These benefits are realized through several mechanisms, including improved gut morphology under disease stress. Healthier gut morphology and improved gut integrity result in improved nutrient uptake and growth benefits, both based on consumption of the disclosed inventive composition. The downstream results are improvements in performance parameters related to gut health including altering gut microbes, feed conversion rates, and body weight gains among others.

The mechanisms of action of the disclosed inventive composition and method are via immune system priming, a form of immune modulation, rather than a direct effect on pathogens, thus there is no risk of treatment resistance being developed. It is to be further noted that while the treatment compound and method of treatment disclosed herein are primarily directed to the improvement of gut health in poultry, the same compound and treatment methodology may effectively be applied to other animal species having similar conditions and issues.

II. Use of Brevundimonas Microbes as an Alternative Treatment for Coccidiosis

In one aspect, the disclosed inventive composition provides an improved prevention and treatment method for a broad variety of diseases including coccidiosis, necrotic enteritis, and other conditions related to gut inflammation via a direct effect on innate and adaptive immune pathways. The disclosed inventive composition also provides an improved prevention and treatment method for other diseases caused by various infectious pathogens including, but not limited to, bacteria, viruses, parasites, and fungi.

Outbreaks of coccidiosis and necrotic enteritis are typically concurrent. It is known that a coccidiosis infection predisposes an animal to necrotic enteritis as the damage from the coccidiosis infection creates an ideal environment in which the C. perfringens, the causative agent in necrotic enteritis, may flourish. The downstream results of these infections are a reduction in performance parameters related to gut health (including altering gut microbes), feed conversion rates, and body weight gains among others. When administered in poultry feed or drinking water, the bioactives of the disclosed inventive composition mitigate these effects of coccidiosis via an enhanced immune response rather than a direct effect on a pathogen, such as the Eimeria parasite.

The mechanisms of action of the disclosed inventive composition and method are via immune system priming rather than a direct effect on pathogens, thus there is no risk of treatment resistance being developed. Due to broad spectrum immune modulation, animal growth and development are enhanced systemically.

III. Natural Feed Composition Containing Brevundimonas for the Promotion of Animal Growth

In another aspect, the disclosed inventive composition generally provides a natural compound for use as a feed ingredient to promote animal growth. The compound of the disclosed inventive composition is combined with conventional feed for administration to animals, such as poultry. Human application is possible, as well. The combination of the disclosed inventive composition and conventional feed works through unique pathways. For example, in healthy birds it serves to enhance growth performance while also priming the immune system to expedite response to a disease challenge should one arise. The disclosed inventive composition is a natural product and thus has no adverse environmental impact.

During the treatment period, the disclosed inventive composition or biomass comprising a culture containing the Brevundimonas bacteria or culture of isolated Brevundimonas bacteria is administered to the animal by way of poultry feed, drinking water, or both. Studies based on the use of animal feed stock including the disclosed inventive composition or biomass revealed improved growth in animals. It was found that the animals, particularly healthy birds, benefited significantly in terms of improved growth efficiency when fed the biomass of the disclosed inventive composition mixed with a conventional diet, such as a corn-soy diet. Feeding healthy chickens (specifically, broiler chickens) a corn/soy diet supplemented with the inventive composition of culture biomass containing Brevundimonas improves growth efficiency of the birds such that the Body Weight Gain (BWG) and Feed Conversion Ratio (FCR) are improved in a statistically significant manner compared to birds fed the same diet without biomass supplementation. It should be understood that while reference herein is made to a conventional diet of corn and soy, the disclosed compound may also be used to advantage in combination with other forms of conventional animal feed, such as, but not limited to, wheat.

In a study involving a natural feed composition derived from cultures containing Brevundimonas for the promotion of animal growth, an analysis of tissues collected from sacrificed birds fed the dietary mixture of the inventive composition and conventional feed suggests that the biomass causes an alteration of signaling in multiple growth-related pathways. These pathways include, but are not limited to, those associated with the activator protein 1 (AP-1), the vascular endothelial growth factor (VEGF), the mitogen-activated protein kinase (MAPK or MAP kinase), Ak strain transforming (Akt), and the neurotrophic tropomyosin-related kinase (NTRK). Evidence supports the conclusion that this alteration represents a beneficial modulation of the various pathways.

The disclosed inventive composition has numerous advantageous applications in humans and animals including but not limited to: (1) improving health without the use of further supplements such as antibiotics, enzymes, probiotics, or antimicrobials and (2) providing an all-natural solution to the need for improved growth rate. The disclosed compound may also have growth-enhancing effects when fed to bovine, porcine, avian, equine, ovine, and caprine species.

IV. Enduring Effects on Coccidiosis Prevention and Treatment Via Animal Feed

A further aspect of the disclosed inventive composition provides an improved long-lasting treatment that is easy to administer and is cost effective for a broad variety of diseases for use in both animals and humans such as, but not limited to coccidiosis, necrotic enteritis, and other conditions. The disclosed method and composition provide both disease prevention and treatment via immune modulation early in broiler life.

The treatment has a lasting effect throughout the entire broiler growth cycle. The composition itself is a natural product and thusly has no adverse environmental impact unlike antibiotic regimens.

When the disclosed compound, an active compound that may be derived from a cell wall component, such as lipopolysaccharide (LPS) or a lipoprotein derived from a gram-negative bacteria, including but not limited to Brevundimonas, is administered to the animal by way of poultry feed, drinking water, or both, the actives of the compound mitigate the effects of coccidiosis well after withdrawal of the active material even in the presence of ongoing coccidiosis exposure. Thus, the approach of the disclosed inventive composition stands in sharp contrast to known and commonly used treatments for which the effect is dependent on the on-going presence of the active agent in the feed. The disclosed compound may also have an enduring effect when fed early to livestock including cattle (bovines), pigs (porcines), sheep (ovines), goats (caprines), rabbits (leporines) and domesticated avians (such as but not limited to poultry).

V. Immune Priming to Accelerate/Enhance Immune Response Through Administration of Natural Immune Modulator

Still a further aspect of the disclosed inventive composition is the provision of a long-term treatment for a broad variety of diseases in both animals and humans. In animals, and particularly in poultry, the treatment method and composition has proven effective against, for example, coccidiosis. The compound is easy to administer and is cost effective. The disclosed method and composition provide both disease prevention and treatment by way of immune priming.

The compound of the disclosed inventive composition is combined with conventional feed for administration to animals, such as poultry starting on Day 1. Human application is possible, as well. The feeding regimen changes the status of the innate (early stage) immune pathways as well as the adaptive (later stage) immune pathways whereby the overall immune system is primed. The result of such immune priming is an accelerated and more robust response when challenged by pathogens including, but certainly not limited to, coccidiosis-causing Eimeria. It is to be understood that the disclosed method and compound are applicable to a wide variety of disease in both animals and humans where immune priming would not only reduce the severity and term of the disease state but may also prevent it entirely.

During the treatment period, the disclosed compound derived from an active molecule of a gram-negative bacteria including but not limited to Brevundimonas is administered to the animal by way of feed, drinking water, or both. The active molecule may be a cell wall component such as lipopolysaccharide (LPS) or a lipoprotein. Thus, the approach of the disclosed inventive composition stands in sharp contrast to known and commonly used disease treatments.

Feeding healthy chickens (specifically, broiler chickens) a corn/soy diet supplemented the inventive composition (e.g., biomass of a culture containing Brevundimonas), improves growth efficiency while simultaneously improving immune response compared to birds fed the same diet without culture biomass supplementation. It should be understood that while reference herein is made to a conventional diet of corn and soy, the disclosed compound may also be used to advantage in combination with other forms of conventional animal feed, such as, but not limited to, wheat.

The disclosed inventive composition has numerous applications in humans and animals including but not limited to functioning as a general booster of immune health, as a dietary supplement, and an overall reduction in the need for antibiotics.

VI. Use of TLR4 Modulator in the Treatment of Coccidiosis

An additional aspect of the disclosed inventive composition is the provision of an effective treatment for a broad variety of diseases through the modulation of the inflammatory response normally associated with the disease. Particularly, the disclosed inventive composition is based on the modulation of the TLR pathways by the disclosed inventive composition, which is capable of selectively modulating the TLR4 and TLR2 signaling pathways.

A non-limiting exemplary use of the disclosed inventive composition as a treatment for disease is its use as a replacement for coccidiostats in the treatment of parasitic infections such as coccidiosis. The modulated inflammatory response has been found to result in improved intestinal morphology including the promotion of intestinal barrier integrity. The improvement in poultry health was achieved without the use of antibiotics. Delivery of the composition is made by oral administration of the active materials mixed into feed or drinking water.

### DESCRIPTION OF THE DRAWINGS

For a more complete understanding of this invention, reference should now be made to the accompanying figures in which:

FIG. 1 is a graph illustrating average pen body weight at Day 1 according to the first study;

FIG. 2 is a graph illustrating enhanced feed conversion ratio in cocci-challenged subject animals fed a composition according to the disclosed inventive concept according to a first study;

FIG. 3 is a graph illustrating feed intake comparison in cocci-challenged subject animals fed a composition according to the disclosed inventive concept according to the first study;

FIG. 4 is a graph illustrating average pen body weight comparison in cocci-challenged subject animals fed a composition according to the disclosed inventive concept according to the first study;

FIG. 5 is a graph illustrating reduction of mortality due to Eimeria infection in cocci-challenged subject animals fed a composition according to the disclosed inventive concept according to the first study;

FIG. 6 is a graph illustrating reduction of an intestinal lesion score of the duodenum in cocci-challenged subject animals fed a composition according to the disclosed inventive concept according to the first study;

FIG. 7 is a graph illustrating reduction of an intestinal lesion score of the ileum in cocci-challenged subject animals fed a composition according to the disclosed inventive concept according to the first study;

FIG. 8 is a graph illustrating reduction of an intestinal lesion score of the ceca in cocci-challenged subject animals fed a composition according to the disclosed inventive concept according to the first study;

FIG. 9 is a graph illustrating restoration/protection of ileum villi cell height in cocci-challenged subject animals fed a composition according to the disclosed inventive concept according to the first study;

FIG. 10 is a graph illustrating restoration/protection of ileum crypt depth in cocci-challenged subject animals fed a composition according to the disclosed inventive concept according to the first study;

FIG. 11 is a graph illustrating restoration/protection of ileum villi height to crypt depth ratios in cocci-challenged subject animals fed a composition according to the disclosed inventive concept according to the first study;

FIG. 12 is a graph illustrating reduction of Eimeria in the duodenum gut in cocci-challenged subject animals fed a composition according to the disclosed inventive concept according to the first study;

FIG. 13 is a graph illustrating reduction of Eimeria in the ileum in cocci-challenged subject animals fed a composition according to the disclosed inventive concept according to the first study;

FIG. 14 is a graph illustrating reduction of Eimeria in the ceca in cocci-challenged subject animals fed a composition according to the disclosed inventive concept according to the first study;

FIG. 15 is a graph illustrating the groups, the test material administered, the inclusion amounts, the group to which the material was fed, the status of coccidiosis challenge, and the lesion scoring dates for the second study;

FIG. 16 is a graph illustrating average pen body weight at Day 1 according to the second study;

FIG. 17 is a graph illustrating feed intake comparison in cocci-challenged subject animals fed a composition according to the disclosed inventive concept according to the second study at Day 28;

FIG. 18 is a graph illustrating enhanced feed conversion ratio in cocci-challenged subject animals fed a composition according to the disclosed inventive concept according to a second study at Days 1-28;

FIG. 19 is a graph illustrating reduction of mortality due to Eimeria infection in cocci-challenged subject animals fed a composition according to the disclosed inventive concept according to the second study at Days 1-28;

FIG. 20 is a graph illustrating average pen body weight gain comparison in cocci-challenged subject animals fed a composition according to the disclosed inventive concept according to the second study;

FIG. 21 is a graph illustrating reduction of an intestinal lesion score of the duodenum in cocci-challenged subject animals fed a composition according to the disclosed inventive concept according to the second study at Day 28;

FIG. 22 is a graph illustrating the coccidia incidence score in the small intestine in cocci-challenged subject animals fed a composition according to the disclosed inventive concept according to the second study at Day 28;

The reference “Significance (P<0.05)” in the figures refers to means within a row without a common superscript are significantly different (P<0.05), as determined by Duncan's New Multiple Range Test (MRT 1955).

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The Compound Used in Treatment

The disclosed method of treatment preferably, but not absolutely, utilizes a compound generally derived from an active compound that may be found in the cell wall of a Gram-negative bacterial strain that is a member of the genus Brevundimonas.

As used herein, “modulator” refers to an activator, an inhibitor, or both. Modulation may be the result of activity by at least one Toll-like receptor (TLR), such as TLR4 or possibly TLR2 as well as other Toll-like receptors. As used herein, the term “inhibitor” refers to a molecule that reduces or attenuates the activity induced by another molecule. By way of example, a compound that might block the LPS-dependent activation of TLR4 present on the surface of immune cells in humans and animals would be regarded as an inhibitor of this particular pathway.

As used herein, the term “culture” is defined as microorganisms (either isolated or in combination) that grow in a liquid medium. Unless expressly stated otherwise, the term “biomass” refers to the microorganism cells (with the liquid culture medium removed). The “biomass” can be wet material or dried material.

Unless expressly stated otherwise, the term “supernatant” is defined as the culture medium in which the biomass is grown that contains excreted compounds from the biomass. Supernatant is obtained by growing biomass in culture medium for an appropriate length of time and then removing the microorganism cells by filtration and/or centrifugation.

Brevundimonas is a Gram-negative aerobic, non-fermenting bacterium that can grow under a variety of conditions. It is capable of metabolically utilizing several natural compounds generated by plants or algae.

Embodiments of the compound used in the treatment of disease as set forth herein include one or more LPS/Lipid A compounds produced by Gram-negative bacterial strains for use as selective modulators of the TLR signaling pathway, such as the TLR4 pathway. The disclosed inventive composition involves any combination of three fundamental steps: (1) the Gram-negative bacteria produces LPS/Lipid A compounds; (2) the LPS/Lipid compounds modulate TLR4 activity through activation or inhibition; and (3) a downstream effect results in enhanced innate and adaptive immune processes, thereby aiding in the treatment of coccidiosis, necrotic enteritis, and other conditions related to gut inflammation.

In an embodiment, the LPS/Lipid A compounds used as selective modulators of the TLR4 signaling pathway are produced from a strain of the genus Brevundimonas. The Brevundimonas strain may be a naturally occurring strain found in an algal biomass.

Accordingly, embodiments of the compound used in the treatment of disease according to the present disclosure are directed to one or more LPS/Lipid A compounds produced by a Gram-negative bacterial strain of the group Brevundimonas for use as selective modulators of the TLR signaling pathway.

The LPS/Lipid A compound employed herein may preferably be obtained from a member of the group comprising but not necessarily limited to any one of Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis by any suitable method. In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable techniques. In some examples, analysis of the structure of the LPS compound is performed using routine methods in the art, including using mass spectrometry, gas chromatography, or both.

The one or more LPS/Lipid A compounds derived from Gram-negative bacterial strains of the genus Brevundimonas, may selectively modulate the TLR4 signaling pathway to alter inflammatory responses and to improve immune health in a variety of uses and applications. In an embodiment, the LPS/Lipid A compound derived from species of the genus Brevundimonas may be incorporated within a feed ingredient to improve gut health of poultry.

The disclosed LPS/Lipid A compound derived from a species of the genus Brevundimonas may be used to improve the health of poultry or other animals through a variety of mechanisms. For example, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. In a further example, the LPS/Lipid A compound may modulate TLR4 through either ligand-dependent or ligand-independent activation. As another example, the LPS/Lipid A compound may act in concert with other TLR agonists to provide a heightened immune response, while reducing the metabolic costs to the host.

The treatment compound is dried biomass containing Gram-negative bacteria of the genus Brevundimonas, or compounds derived therefrom, provided in drinking water or as animal feed. In animal feed, once the biomass and feed additive are combined to the preferred premix level, the combined batch is poured or administered evenly into a ribbon mixer containing finished feed. The combined batch is preferably provided in an amount of between about 100.0 g dried Brevundimonas per ton of finished feed and about 150.0 g per ton of finished feed, is more preferably provided in an amount of between about 120.0 g per ton of finished feed and 130.0 g per ton of finished feed and is most preferably though not exclusively provided in an amount of about 125.0 g dried Brevundimonas per ton of feed with good efficacy without being wasteful. As an alternative, the combined batch may be provided in an amount of between about 100.0 g dried Brevundimonas per ton of finished feed and about 300.0 g per ton of finished feed, is more preferably provided in an amount of between about 150.0 g per ton of finished feed and 250.0 g per ton of finished feed, and is most preferably though not exclusively provided as an alternative in an amount of about 200.0 g dried Brevundimonas per ton of feed with good efficacy without being wasteful. As an active fraction or isolated molecule, the amounts administered are less than the amounts needed for whole cells in that the amount required for disease prevention and treatment depend on the potency of the material. For example, where purified LPS is used in feed the ratio is between about 0.1 g per ton of finished feed and about 3.0 g per ton of finished feed with the preferred range being between about 0.16 g per ton of finished feed and about 2.5 g per ton of finished feed. Where the active is administered in water, where a supernatant is the source from an algal culture, the amount is between about 0.1 and 0.3 mL per liter of drinking water with 0.2 mL per liter being most preferred. Where a semi-pure LPS fraction is utilized, the amount is between about 1.0 and 2.0 mg/L with the preferred amount being 1.5 mg/L. Where a purified LPS is utilized, the amount is between about 0.001-0.03 mg/L with between about 0.002-0.02 g/L being most preferred.

Investigations

Investigations were undertaken to determine the response and efficacy of a feed ingredients containing Brevundimonas biomass incorporated at a specific amount into a commercial-type corn-soybean diet and fed to floor-pen raised broilers challenged with Eimeria to cause coccidiosis. The investigations were undertaken over a 28-42 day period, from Day 0 to Day 28 or Day 0 to Day 42. Twenty investigations were performed under a wide range of test conditions and configurations designed to represent the broad range of conditions expected in commercial poultry houses. Several of the investigations were performed by administering biomass from the isolated active Brevundimonas bacterial strain. All other investigations involved administering culture biomass containing the active bacterial strain as the test material.

Investigations were conducted in both battery cages and floor pens, using both mash and pelleted feed rations using appropriate numbers of birds and replicates to validate statistically significant findings (P<0.05). In most investigations, a disease challenge involving three species of Eimeria (E. tenella, E. maxima, and E. acervulina) was applied, but the timing of the challenge was varied to validate efficacy under different infection scenarios. Investigation endpoints included performance parameters (e.g., feed intake, body weight gain, FCR), disease endpoints (e.g., mortality, lesion score, intestinal morphology, parasite enumeration). While a majority of investigations were conducted over the full 42-day broiler grow-out period, several investigations were truncated to 28-days in order to streamline the product development cycle after validating that the results from 28-day investigations are fully representative and predictive of results for full-length investigations. Two specific studies selected from the full research program are discussed here.

First Study—Treatment Method

Chicks were obtained within twelve hours of hatching from fecal contaminated flocks at a commercial hatchery on Day 0 (hatch and placement day). Three-hundred mixed-sex broiler chicks (50:50 sex ratio) were randomly assigned on Day 0 by individual weights to each of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets as pellets (crumbled pellets for Days 1-14). Birds were fed their respective treatment diets ad libitum from day of hatch to 28 days of age. (The typical average market age is 42 days.) Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds assigned to challenge test groups received oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge—On Day 7, all birds assigned to challenged test groups received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

First Study Evaluation

Differences between groups were evaluated at P<0.05, employing Treatment x Replicate RCB (Randomized Complete Block) design. Particularly and as noted, “Significance (P<0.05)” refers to means for groups not sharing common letter designations in the figures which indicates significant difference (P<0.05) between said groups, as determined by Duncan's New Multiple Range Test (MRT 1955).

The relevant evaluation results from the first study are set forth in the following chart and are referenced below by individual graphically represented figures:

| | Feed | | | | | | |
|---|---|---|---|---|---|---|---|
| | Conversion | % | Avg Pen | | Lesion | Lesion | Lesion |
| | Ratio | Mortality | Body | Feed Intake | Score | Score | Score |
| | Corrected | Days 1- | Weight (g) | (g/bird/day) | Duodenum | Ileum | Ceca |
| Means | Days 1-28 | 28 | Day 28 | Day 28 | Day 28 | Day 28 | Day 28 |
| 1. No Tx, No | 1.527 | 1.852 | 1418.47 | 88.502 | 0.950 | 0.067 | 0.075 |
| Challenge | | | | | | | |
| 2. No Tx, | 1.595 | 17.593 | 1365.56 | 98.530 | 2.483 | 2.525 | 2.492 |
| Cocci | | | | | | | |
| 3. Coban, | 1.527 | 1.852 | 1430.88 | 89.499 | 0.525 | 0.433 | 0.583 |
| Cocci | | | | | | | |
| 4. Brev, | 1.536 | 3.241 | 1446.26 | 91.538 | 0.450 | 0.500 | 0.483 |
| Cocci | | | | | | | |
| Significance | | | | | | | |
| (P < 0.05) | | | | | | | |
| 1. No Tx, No | a | a | a | a | b | a | a |
| Challenge | | | | | | | |
| 2. No Tx, | b | b | b | b | c | c | c |
| Cocci | | | | | | | |
| 3. Coban, | a | a | a | a | ab | b | b |
| Cocci | | | | | | | |
| 4. Brev, | a | a | a | a | a | b | b |
| Cocci | | | | | | | |

Benefits of Disclosed Inventive Composition in Animal Growth Performance

The disclosed inventive composition offers several benefits not seen in known feed compositions. The results from the 20 studies performed demonstrate that the actives of the disclosed inventive composition consistently improve the efficiency with which broiler chickens convert feed into body mass gains in the presence of a disease challenge. More specifically as illustrated in the representative study described above in conjunction with the attached figures, Eimeria-challenged birds ingesting the actives of the disclosed inventive composition consistently show statistically significant improvements in FCR compared to non-treated, cocci-challenged birds. In addition to deliberate Eimeria inoculation early in life, studies were conducted on built-up litter providing ongoing exposure to Eimeria and other environmental pathogens during the course of the studies. Despite this, the FCR status of birds fed the disclosed inventive composition was consistently found to be not statistically different from unchallenged birds and challenged birds treated with leading ionophore products commonly used in poultry production, such as Salinomycin, Coban® (Monensin, USP), Elanco Animal Health, and Maxiban® (Narasin and nicarbazin), Elanco Animal Health. These improvements in FCR are reliably shown to be due to increases in body weight and not due to increased feed intake.

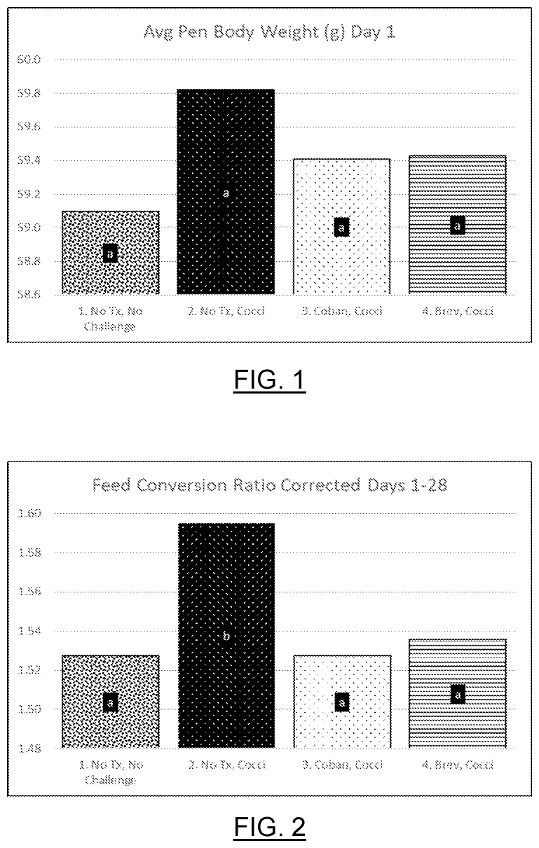

Day 1 Body Weight Evaluation—Referring to FIG. 1, Average Body Weight at Day 1 is illustrated. Body weights are not statistically different among groups at this stage, ranging from a high weight of 59.096 g for birds assigned to the unchallenged and untreated groups to 59.821 g for birds assigned to the challenged and untreated groups:

| | Avg Pen |
|---|---|
| | Body |
| | Weight |
| Means | (g) Day 1 |
| 1. No Tx, No Challenge | 59.096 |
| 2. No Tx, Cocci | 59.821 |
| 3. Coban, Cocci | 59.407 |
| 4. Brev, Cocci | 59.429 |
| Significance (P <0.05) | |
| 1. No Tx, No Challenge | a |
| 2. No Tx, Cocci | a |
| 3. Coban, Cocci | a |
| 4. Brev, Cocci | a |

Feed Conversion Rate—As set forth in FIG. 2 which is a graph illustrating enhanced feed conversion ratio in cocci-challenged subject animals fed a composition according to the disclosed inventive concept, mortality-corrected FCR was determined from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Growth performance as measured by FCR for the group fed the disclosed inventive composition is dramatically improved compared to the challenged/untreated group and not statistically different from the unchallenged/untreated control group or the challenged/Coban® treated group.

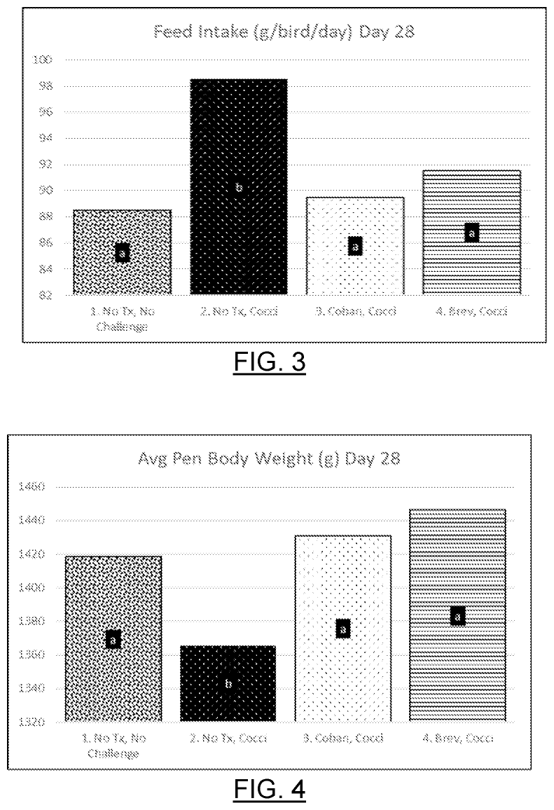

Feed Intake—As set forth in FIG. 3 which is a graph illustrating feed intake comparison in subject animals fed a composition according to the disclosed inventive concept, feed intake was measured from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, feed intake increased significantly in challenged/untreated birds. In contrast, feed intake of challenged birds receiving the immune modulating actives of the disclosed composition was not significantly different than that of the unchallenged/untreated control group or the challenged/Coban® treated group.

Body Weight—As set forth in FIG. 4 which is a graph illustrating average pen body weight comparison in subject animals fed a composition according to the disclosed inventive concept, average pen body weight was measured from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, body weight was significantly decreased in challenged/untreated birds compared to the other groups. In contrast, body weight of challenged birds receiving the immune modulating actives of the disclosed composition was not significantly different than that of the unchallenged/untreated control group or the challenged/Coban® treated group.

Mortality—Across all 20 studies, actives of the disclosed composition consistently reduced mortality in Eimeria-challenged broiler chickens, resulting in mortality levels not statistically different from those observed in unchallenged control groups and challenged birds treated with leading ionophore products commonly used in poultry production.

As set forth in FIG. 5 which is a graph illustrating reduction of mortality due to Eimeria infection in subject animals fed a composition according to the disclosed inventive concept, the percent mortality was calculated from day of hatch through Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Mortality in the challenged/untreated group was approximately 17% indicating that the Eimeria challenge resulted in significant disease. In contrast, mortality of challenged birds receiving the immune modulating actives of the disclosed composition was held to a level not significantly different than that of the unchallenged/untreated control group or the challenged/Coban® treated group.

Protection of Gut

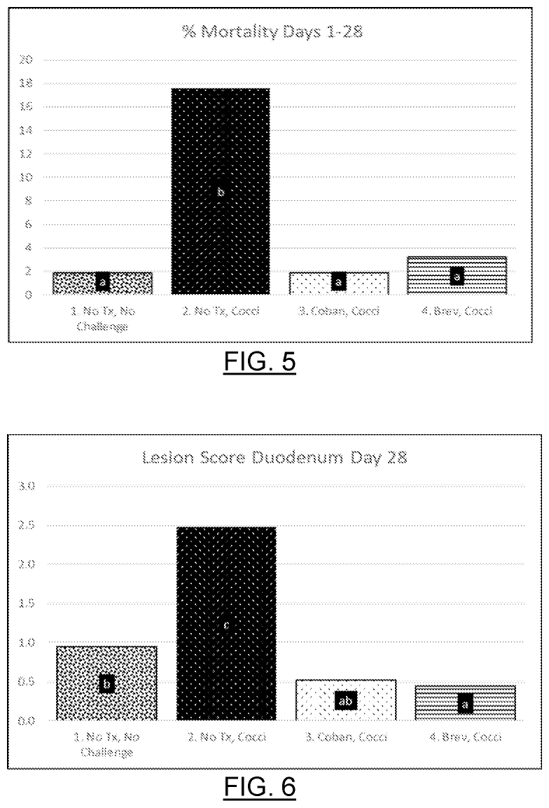

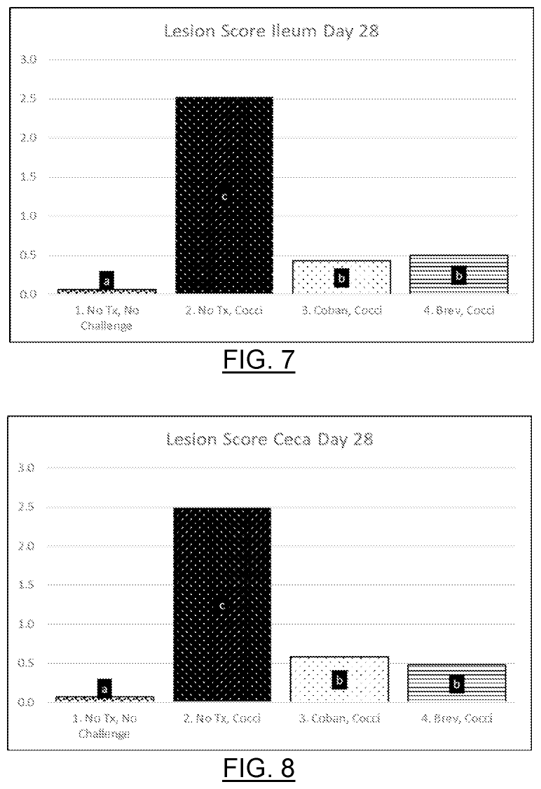

Lesion Scores—Another primary benefit of actives of the disclosed composition is reduced presence and severity of coccidia lesions and damage to the intestinal lining in Eimeria-challenged broiler chickens. Lesion scores in the duodenum, ileum, and ceca are consistently decreased in birds ingesting actives of the disclosed composition, to the same extent as unchallenged control groups and challenged birds treated with leading ionophore products commonly used in poultry production. Lesion score data for the duodenum are provided in FIG. 6. Similar trends were observed in the ileum and ceca as set forth in FIGS. 7 and 8 respectively.

As set forth in FIG. 6 which is a graph illustrating reduction of intestinal lesion score in subject animals fed a composition according to the disclosed inventive concept, intestinal lesion score (Johnson and Reid method) was determined in the duodenum on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). As expected, the challenged/untreated group exhibited significantly more lesions due to the Eimeria challenge when compared to the unchallenged control. The challenged/composition-fed group showed no such increase in lesion score. In fact, the lesion score of the challenged/composition-fed group was numerically and statistically lower than that of the unchallenged control group and not significantly different compared to the challenged birds receiving Coban®. Similar lesion score results were found in the ileum as illustrated in FIG. 7 and in the ceca as illustrated in FIG. 8.

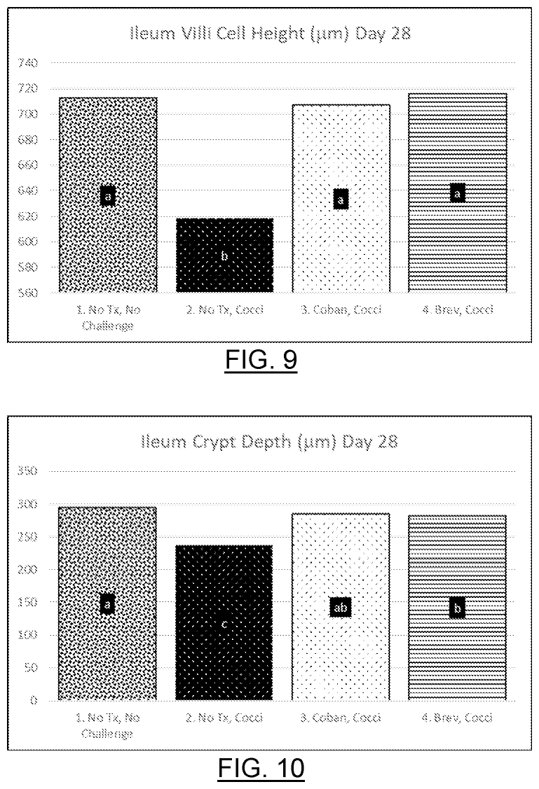

Intestinal Morphology—The immune modulating actives of the disclosed composition also demonstrate positive effects on gut morphology, including restoring or protecting villi height and crypt depth in the ileum following a cocci challenge (FIGS. 9 and 10).

As set forth in FIG. 9 which is a graph illustrating restoration/protection of ileum cell villi height in subject animals fed a composition according to the disclosed inventive concept, cell villi height in the ileum was measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). The detrimental effects of coccidiosis are clearly seen in the challenged/untreated group which had significantly reduced villi height compared to the unchallenged control group. In contrast, there is no significant difference between the cell villi height measured in birds treated with the disclosed composition as compared to the unchallenged control or in challenged birds treated with Coban®.

As set forth in FIG. 10 which is a graph illustrating restoration/protection of ileum crypt depth in subject animals fed a composition according to the disclosed inventive concept, ileum crypt depth was measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Crypt depth in birds fed the actives of the disclosed composition was significantly improved compared to the challenged/untreated group and was not statistically different than for challenged birds treated with Coban®.

As set forth in FIG. 11 which is a graph illustrating restoration/protection of ileum villi height to crypt depth ratios in subject animals fed a composition according to the disclosed inventive concept, which were measured on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Crypt depth ratios in birds fed the actives of the disclosed composition was significantly improved compared to the challenged/untreated group and was only slightly greater than for challenged birds treated with Coban®.

Secondary Benefits of the Inventive Composition

Reduction of Oocysts

Multiple studies demonstrate that the actives of the disclosed composition reduce E. acervulina in the loop of the small intestine area, E. maxima in the jejunum, and E. tenella in the ceca.

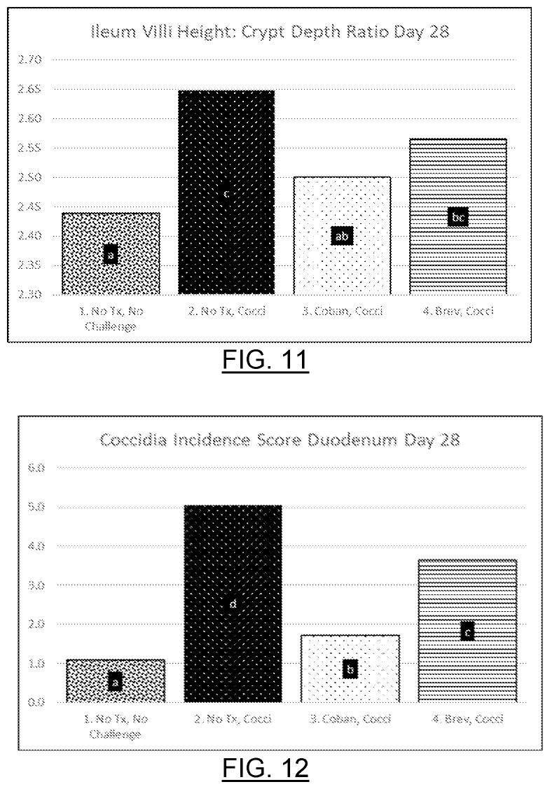

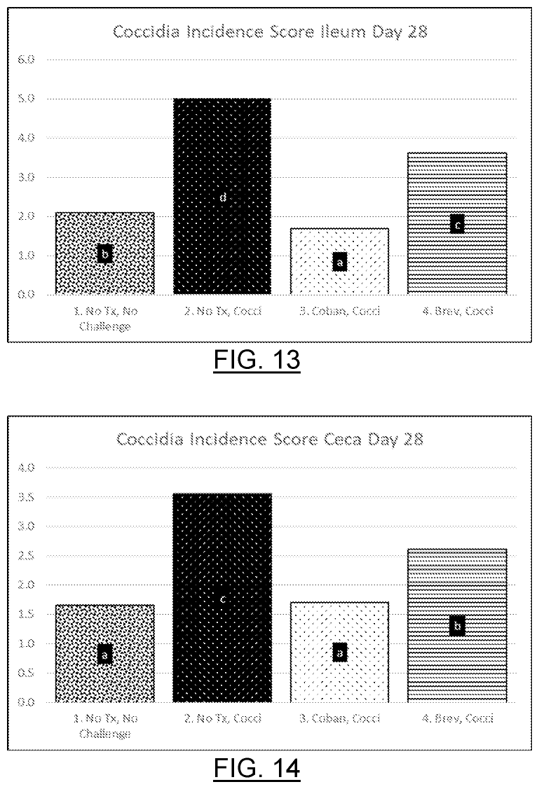

As set forth in FIGS. 12, 13, and 14 which are graphs illustrating reduction of Eimeria in the broiler gut in subject animals fed a composition according to the disclosed inventive concept, coccidia count scores in the duodenum, the ileum, and the ceca respectively are shown confirming trends for all intestinal regions. The coccidia count score was determined on Day 28 for control birds receiving no Eimeria challenge and no treatment (first bar) and birds challenged with Eimeria on Day 7 but untreated (second bar), challenged and treated with Coban® (third bar), or challenged and treated with the immune modulator of the disclosed composition (fourth bar). Actives of the disclosed composition provided a significant reduction in the coccidia score relative to challenge/untreated birds but did not reduce the score to levels comparable with either the unchallenged control or the challenged birds treated with Coban®.

Second Study

A second study was undertaken to determine the response and efficacy of a dried bacterial biomass feed ingredient incorporated at a specific amount into a commercial-type corn-soybean diet and fed to floor-pen raised broilers. The second study was undertaken over a 28-day period, from Day 1 to Day 28.

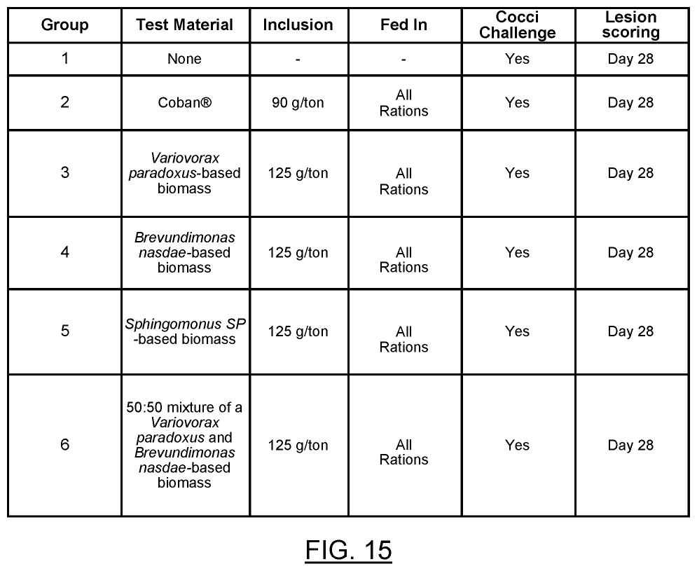

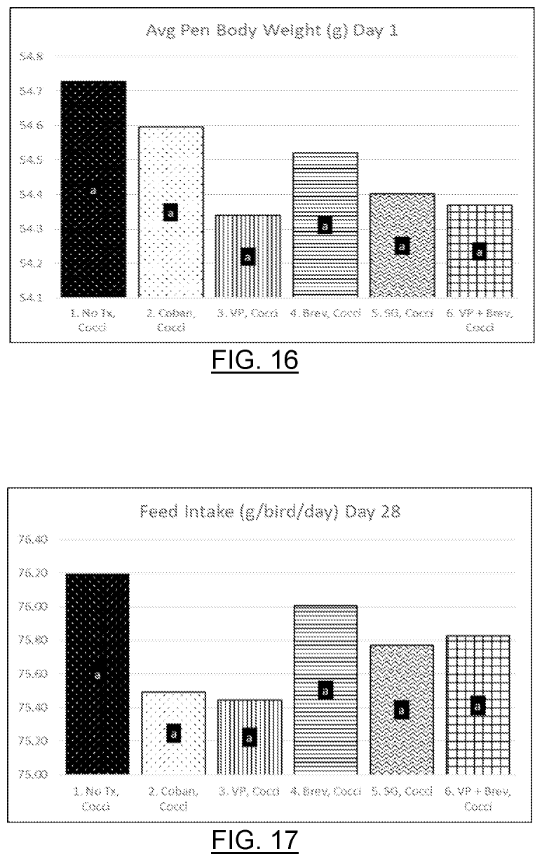

The animals were raised under a disease challenge environment (cocci-challenge+built-up litter). For the study and as set forth in FIG. 15, six groups were established, including a first group that received no feed additive ingredient, a second group treated with the anti-coccidia drug Coban® (Elanco), a third group fed a Variovorax-based biomass, a fourth group fed a Brevundimonas-based biomass, a fifth group fed a Sphingomonas-based biomass, and a sixth group fed a 50:50 mixture of a Variovorax and Brevundimonas-based biomass. The feed included corn and soybean meal rations with normal nutritional formulations. No coccidiostat (except for treatment with Coban® provided to the second group) or ABF (antibiotic free products) were administered during the study. No coccidiosis vaccine was administered at the hatchery or during the course of the study.

Second Study—Treatment Method

A total of 1,800 mixed sex broiler chicks were obtained within twelve hours of hatching from fecal contaminated flocks at a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study.

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

All birds received nutritionally adequate diets as pellets (crumbled pellets for Days 1-14). Birds were fed their respective treatment diets ad libitum from day of hatch to 28 days of age. (The typical average market age is 42 days.) Birds were raised on built-up litter to further mimic stress conditions typically experienced in poultry production.

All diets were offered ad libitum without restrictions to full-fed consumption, except for an 8-hour fasting period prior to cocci-challenge on Day 7 when all birds assigned to the challenge test groups received oocyst-inoculated feed containing a mixture of Eimeria acervulina, Eimeria maxima, and Eimeria tenella. Dietary requirements for protein, lysine, methionine, methionine+cystine, arginine, threonine, tryptophan, total phosphorus, available phosphorus, total calcium, dietary sodium, and dietary choline were met by adjusting the concentrations of corn and soybean meal ingredients, as well as other minor ingredients commonly used in poultry production.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass).

Cocci-Challenge—On Day 7, all birds assigned to the challenge test groups received oocyst-inoculated feed containing a mixture of Eimeria acervulina, Eimeria maxima, and Eimeria tenella. Adequate feed was precisely weighed and provided to birds to consume at the rate of 100% fill-capacity on average. Prior to the challenge, all birds were starved for eight hours. Inoculated feed was provided to the birds. Following a specific time, all remaining inoculated feed was removed and weighed to assure equal consumption per pen and per bird. The quantity of feed (both placed and withdrawn) was recorded on each pen's feed record.

Second Study Evaluation

Differences between groups were evaluated at P<0.05, employing Treatment x Replicate RCB (Randomized Complete Block) design. Particularly and as noted, “Significance (P<0.05)” refers to means within a column without a common superscript are significantly different (P<0.05), as determined by Duncan's New Multiple Range Test (MRT 1955).

The relevant evaluation results from the second study are set forth in the following chart and are referenced below by individual graphically represented figures:

| | | | | | | Coccidia |
|---|---|---|---|---|---|---|
| | Feed | | | | | Incidence |
| | Conversion | | Avg Pen | | Lesion | Score |
| | Ratio | % | Body | Feed Intake | Score | Small |
| | Corrected | Mortality | Weight (g) | (g/bird/day) | Duodenum | Intestine |
| Means | Days 1-28 | Days 1-28 | Day 28 | Day 28 | Day 28 | Day 28 |
| 1. No Tx, Cocci | 1.498 | 15.000 | 1294.183 | 76.196 | 2.075 | 1.683 |
| 2. Coban, Cocci | 1.403 | 3.750 | 1443.182 | 75.493 | 0.317 | 0.325 |
| 3. VP, Cocci | 1.461 | 10.833 | 1339.628 | 75.446 | 1.508 | 1.300 |
| 4. Brev, Cocci | 1.409 | 5.417 | 1433.982 | 76.005 | 1.033 | 0.958 |
| 5. SG, Cocci | 1.456 | 7.917 | 1375.182 | 75.772 | 1.325 | 1.200 |
| 6. VP + Brev, Cocci | 1.435 | 5.833 | 1405.136 | 75.826 | 0.942 | 1.017 |
| Significance | | | | | | |
| (P < 0.05) | | | | | | |
| 1. No Tx, Cocci | d | c | d | a | e | d |
| 2. Coban, Cocci | a | a | a | a | a | a |
| 3. VP, Cocci | c | b | c | a | d | c |
| 4. Brev, Cocci | ab | a | a | a | b | b |
| 5. SG, Cocci | c | ab | bc | a | c | c |
| 6. VP + Brev, Cocci | bc | a | ab | a | b | b |

As noted above, the test subjects were divided into six groups. Members of the first group received no feed additive ingredient in its food regimen. Members of the second group were given feed that included the anti-coccidia drug Coban® (Elanco) according to the manufacturer's instructions. Members of the third group were given feed containing a Variovorax-based biomass. Members of the fourth group were given feed containing a Brevundimonas-based biomass. Members of the fifth group were given feed containing a Sphingomonas-based biomass. Members of the sixth group were given feed containing a 50:50 mixture of a Variovorax and Brevundimonas-based biomass.

All data points were analyzed at the 5% level of probability, including composite weighted average of entire pen, feed:gain and mortality. Birds were evaluated in terms of physical live performance and digestive health. The six groups were evaluated over a 28-day period for body weight, feed conversion ratio (FCR) (corrected for mortality), mortality, body weight gain, and average weight gain.

Day 1 Body Weight Evaluation—Referring to FIG. 16, Average Body Weight at Day 1 is illustrated. Body weights are not statistically different among groups at this stage, ranging from a high weight of 54.727 g for Group 1 to a low weight of 54.339 g for Group 3:

| | Avg Pen |
|---|---|
| | Body |
| | Weight (g) |
| Means | Day 1 |
| 1. No Tx, Cocci | 54.727 |
| 2. Coban, Cocci | 54.595 |
| 3. VP, Cocci | 54.339 |
| 4. Brev, Cocci | 54.521 |
| 5. SG, Cocci | 54.402 |
| 6. VP + Brev, Cocci | 54.369 |
| Significance | |
| (P <0.05) | |
| 1. No Tx, Cocci | a |
| 2. Coban, Cocci | a |
| 3. VP, Cocci | a |
| 4. Brev, Cocci | a |
| 5. SG, Cocci | a |
| 6. VP + Brev, Cocci | a |

Feed Intake—The graph set forth in FIG. 17 illustrates a feed intake comparison in subject animals fed a composition according to the disclosed inventive concept. Feed intake was measured at Day 28 for Group 1 (first bar), for Group 2 (second bar), for Group 3 (third bar), for Group 4 (fourth bar), for Group 5 (fifth bar), and for Group 6 (sixth bar). Feed intake was highest in challenged/untreated birds. However, there were no statistical differences in feed intake for any of the groups.

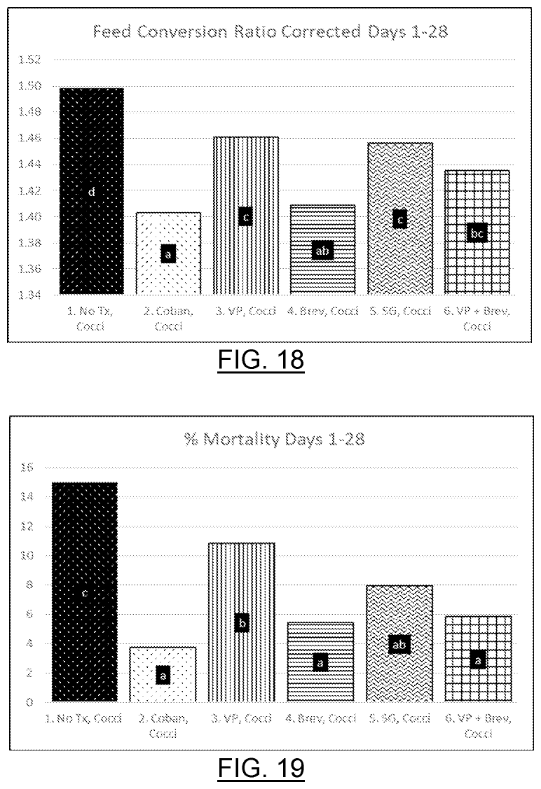

Feed Conversion Rates Corrected—FCR was recorded on Days 1-28 as shown in FIG. 18. Group 2, which received feed that included Coban®, had the lowest FCR, while Group 4, which received feed containing a Brevundimonas-based biomass had the second lowest rate. Starting on Day 8 and continuing throughout the study duration, improvements in FCR for Group 4 were not statistically different from Group 2, demonstrating that the Brevundimonas-based biomass in feed performed as well as a long-standing, commercially available coccidiosis treatment, such as Coban®. Groups 2 and 4 had statistically lower FCR compared to Group 1, which received no feed additive ingredient in its food regimen.

Mortality Percentage—Mortality rates were recorded on Days 1-28 as illustrated in FIG. 19. Mortality rates were lowest for members of the second group, which received feed that included Coban® and for members of the fourth group, which received feed containing a Brevundimonas-based biomass as well as for Group 6, which received feed containing a 50:50 mixture of a Variovorax and Brevundimonas-based biomass.

Average Pen Body Weight—Average pen body weight was recorded on Day 28 and is set forth in FIG. 20. Weight gain was most pronounced for the second group, which received feed that included Coban® but was followed closely by weight gain in members of the fourth group, which received feed containing a Brevundimonas-based biomass. Members of Group 6, which received feed containing a 50:50 mixture of a Variovorax and Brevundimonas-based biomass also showed noteworthy weight gain.

Lesion Scores—Gross necropsy and lesion scoring were performed. Birds were selected, sacrificed, weighed, and examined for the presence and degree of duodenal and coccidia lesions. Damage scores were assessed and recorded.

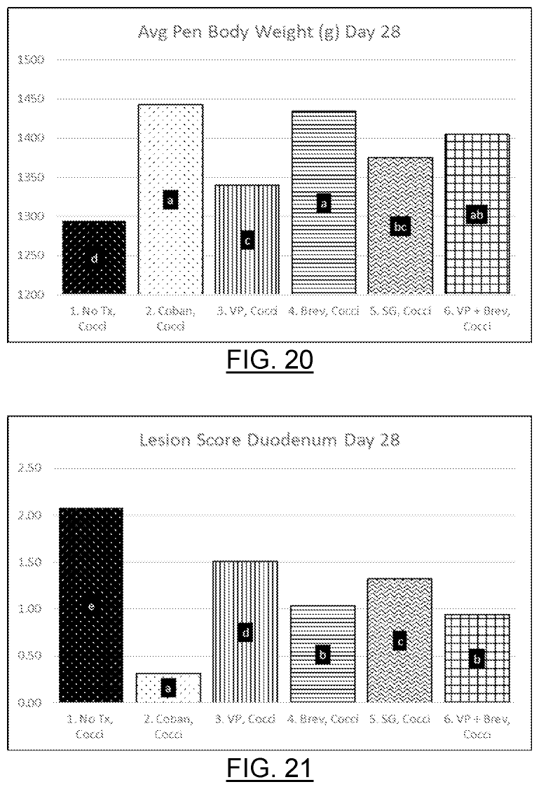

With respect to the duodenum lesion scores for Day 28, the lowest score was evident with the second group, which received Coban®, followed by groups 4 given feed containing a Brevundimonas-based biomass and 6 given feed containing a 50:50 mixture of a Variovorax and Brevundimonas-based biomass. These results are illustrated in FIG. 21.

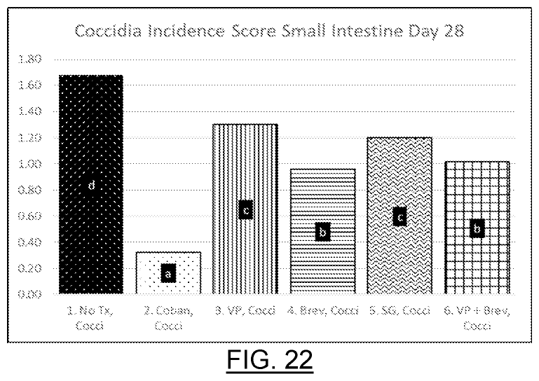

With respect to the coccidia lesion incidences score of the small intestine for Day 28, the lowest score was again experienced by the second group, a statistically significant reduction compared to all other groups, followed by the fourth and sixth groups. These results are illustrated in FIG. 22.

Results—in General

The disclosed inventive feed additive containing a Brevundimonas-based biomass consistently promoted improved growth performance in broilers receiving a coccidiosis challenge that was not statistically different from that observed for birds given feed that included the anti-coccidia drug Coban®. This outcome holds true over all assessed growth performance characteristics including body weight, feed conversion ratio (FCR), mortality, body weight gain and average weight gain. The positive results obtained with the inventive composition were so observed without risk of drug resistance developing as is often the risk with the use of anti-coccidia drugs.

I. Results—Agents and Method for Improving Gut Health

The disclosed inventive composition demonstrates improved live performance and digestive health parameters for weight gain, feed efficiency, mortality, intestinal villi cell height, crypt depth, villi height to crypt depth ratio, and intestinal coccidiosis across all age ranges. The disclosed inventive composition is effective in ameliorating the physical effects of environmental stress on live performance that are typically experienced in poultry production.

The disclosed inventive composition demonstrates a positive impact on the gut health of boilers through improved gut morphology under disease stress by improving nutrient uptake and eventual bird growth and overall improved gut integrity. The treatment method and compound may have benefits that go beyond the poultry industry to other animals and possibly to humans.

II. Results—Use of Brevundimonas Microbes as an Alternative Treatment for Coccidiosis

In an additional study which considered the use of Brevundimonas microbes as an alternative treatment for coccidiosis, analysis of tissue from broiler chickens provided a feed mixture that included the disclosed compound demonstrated a significant maturing of innate immune pathways when compared with sacrificed birds fed a conventional feed mixture without the disclosed compound. Also, in general, kinomic analysis of tissue from the birds provided a feed mixture that included the disclosed compound demonstrated significant enhancement of adaptive immune pathways.

Furthermore, analysis of the broiler tissues from chickens fed the treatment compound according to the regimen set forth above, as well as those not fed the treatment compound, verified that the treated chickens demonstrated an increase in adaptive immune response indicating a more immunologically competent or developed immune system. Significantly, the analysis indicated that treatment with the disclosed compound resulted in a more adaptive-biased immune system in the supplemented birds and further demonstrated an alteration of immunometabolism in gut, liver, and muscle tissue by priming the animal for a more rapid and robust immune response compared to birds receiving a standard, unfortified diet. Comparative analysis of tissues of chickens fed the treatment compound and those not fed the treatment compound verified that the treated chickens demonstrated an altered immune response consistent with the effects of TLR4 modulation.

Advantageously, studies showed that the identified immunomodulatory adaptation does not come at the cost of impaired growth and development, rather metabolic efficiency was facilitated in the latter part of life. By following the method and treatment of the disclosed inventive concept, a more rapid, adaptive immune response that is comparable to a response usually seen in an older bird is possible in young birds.

III. Results—Natural Feed Composition Containing Brevundimonas for the Promotion of Animal Growth

In general, analysis of the results supports the conclusion that inclusion of the innovative compound as part of a conventional diet leads to a significant increase in growth and weight gain when compared to the control flock.

Moreover, following treatment with the disclosed compound on selected birds, samples of both treated and non-treated birds were examined by gross necropsy which included internal examination. Kinomic analysis of tissues collected from sacrificed birds fed the dietary mixture of the inventive composition and conventional feed confirmed that the biomass alters multiple growth-related pathways as proposed, thus initiating pathway activation.

Further analysis demonstrates that the birds consuming the supplemented diet converted feed into body mass more efficiently than the birds fed the control diet. Birds receiving the diet supplemented with the inventive composition had statistically significant decrease in FCR compared to the untreated control group in the given studies. Perhaps most significantly, while there were no differences in overall carcass yield, the total breast weight, as well as the Pectoralis major (breast fillets) and Pectoralis minor (tender) weights, were increased in the group fed the diet supplemented with the inventive composition disclosed herein.

IV. Results—Enduring Effects on Coccidiosis Prevention and Treatment Via Animal Feed

The use of the innovative compound in the treatment of coccidiosis-challenged poultry demonstrates an enduring effect on the coccidiosis prevention and treatment by delivery through animal feed whereby the positive effects of treatment persist well beyond the time of product withdrawal unlike the loss of efficacy noted immediately after withdrawal of conventional anticoccidial products. Overall, analysis of birds fed the test material for the first 14 days of life demonstrated changes in the immune system consistent with a bolstered innate immune response thereby providing enduring effects over the life of the animal.

Notably, a significant decrease in the presence and degree of coccidia lesions and damage to the intestinal lining typically experienced following coccidiosis infection was noted.

Upon examination of the sacrificed birds, it was found that the average lesion scores throughout the intestine of poultry treated with the disclosed composition were consistently lower than the scores of sacrificed untreated disease-challenged birds. Ileum villi cell height, crypt depth, and the villus height to crypt depth ratio generally showed improvement. Particularly, ileum villi cell height and crypt depths in birds fed the inventive formulations disclosed herein were competitive with coccidiosis-challenged birds treated with conventional antibiotics.

The average oocyst count of the duodenum, mid-gut, and cecum of sample poultry given feed or water having the disclosed inventive composition were lower than the scores of sacrificed untreated disease-challenged birds. The groups treated with the inventive composition had improved mortality compared to challenged, untreated groups.

The improvement of the overall health of disease-challenged poultry as a result of being given feed or water containing the disclosed inventive composition was achieved without the use of antibiotics. Overall the inventive composition demonstrates a cost-effective and practical approach to the treatment of disease states in animals.

What is claimed:

1. A composition for the prevention and treatment of disease in animals, the composition comprising effective amounts of a feed or drink ingredient including an active molecule derived from a Gram-negative bacterium of the genus Brevundimonas.

2. The composition of claim 1 wherein the active molecule is a cell wall component.

3. The composition of claim 2 wherein the cell wall component is taken from the group consisting of a lipopolysaccharide and a lipoprotein.

4. The composition of claim 1 wherein the bacterium is selected from the group consisting of Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis.

5. The composition of claim 1 wherein the composition is effective in the prevention and treatment of disease in poultry.

6. The composition of claim 1 wherein the composition is administered in an amount of between about 100.0 g dried Brevundimonas per ton of finished feed and about 300.0 g per ton of finished feed where whole bacterial cells are utilized.

7. The composition of claim 1 wherein the compound is administered in an amount of between about 0.1 g per ton of finished feed and about 3.0 g per ton of finished feed where purified LPS are utilized.

8. The composition of claim 1 wherein the compound is administered in an amount of between about 0.1 mL per liter of drinking water and about 0.3 mL per liter of drinking water when provided in drinking water.

9. A composition for the prevention and treatment of disease in animals, the composition comprising effective amounts of a feed or drink ingredient including an extracted active fraction or a purified active molecule obtained from a Gram-negative bacterium of the genus Brevundimonas.

10. The composition of claim 9 wherein the extracted active fraction or purified active molecule is a cell wall component.

11. The composition of claim 10 wherein the cell wall component is taken from the group consisting of a lipopolysaccharide and a lipoprotein.

12. The composition of claim 9 wherein the bacterium is selected from the group consisting of Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis.

13. The composition of claim 9 wherein the composition is effective in the prevention and treatment of disease in poultry.

14. The composition of claim 9 wherein the composition is administered in an amount of between about 100.0 g dried Brevundimonas per ton of finished feed and about 300.0 g per ton of finished feed.

15. A selective modulator of one or more TLR pathways comprising an extracted or a purified active molecule derived from a Gram-negative bacterium of the genus Brevundimonas wherein the Gram-negative bacteria is an agonist of one or more TLR pathway.

16. The composition of claim 15 wherein the extracted or purified active molecule is a cell wall component.

17. The composition of claim 16 wherein the cell wall component is taken from the group consisting of a lipopolysaccharide and a lipoprotein.

18. The selective modulator of claim 15 wherein the bacterium is selected from the group consisting of Brevundimonas vesicularis, Brevundimonas nasdae, Brevundimonas intermedia, Brevundimonas aurantiaca, Brevundimonas mediterranea, Brevundimonas albigilva, and Brevundimonas huaxiensis.

19. The composition of claim 15 wherein the composition is effective in the prevention and treatment of disease in poultry.

20. A method for treating an animal for clinical or subclinical disease through modulation of one or more TLR pathways, the method comprising
identifying a Gram-negative bacterium;
extracting an active molecule from the Gram-negative bacterium;
purifying the extracted molecule to form a LPS/Lipid A compound;
adding a pharmaceutically effective amount of the LPS/Lipid A compound to animal feed or drink to form a treatment composition; and
administering a pharmaceutically effective amount of the treatment composition to the animal at its earliest life stage.

21. The method of claim 20 wherein the Gram-negative bacterium is of the genus Brevundimonas.

22. The composition of claim 20 wherein the active molecule is a cell wall component taken from the group consisting of a lipopolysaccharide and a lipoprotein.

23. The method of claim 20 wherein the treatment composition is administered to the subject animal beginning on Day 1 of its life thereby preventing parasitic disease through immune modulation.
